(19) United States
(12) Patent Application Publication
(10) Pub. No.: US 2024/0170155 A1
(43) Pub. Date: May 23, 2024

(54) STRESS ESTIMATION DEVICE, STRESS ESTIMATION METHOD, AND STORAGE MEDIUM

(71) Applicant: NEC Corporation, Minato-ku, Tokyo (JP)

(72) Inventors: Kei SHIBUYA, Tokyo (JP); Azusa Furukawa, Tokyo (JP); Masanori Tsujikawa, Tokyo (JP)

(73) Assignee: NEC Corporation, Minato-ku, Tokyo (JP)

(21) Appl. No.: 18/284,906

(22) PCT Filed: Apr. 2, 2021
(86) PCT No.: PCT/JP2021/014356

§ 371 Date: Sep. 29, 2023

(51) Int. Cl. G16H 50/30; A61B 5/00; A61B 5/16; A61B 5/369

(52) U.S. Cl. CPC G16H 50/30; A61B 5/165; A61B 5/7267; A61B 5/369

(57) ABSTRACT

A stress estimation device 1X mainly includes a static attribute information acquisition means 14X, an observation information acquisition means 15X, and a stress estimation means 18X. The static attribute information acquisition means 14X acquires static attribute information regarding a static attribute of a subject. The observation information acquisition means 15X acquires observation information that is information obtained by observing the subject. The stress estimation means 18X calculates a stress estimate value, which is an estimate value representing a degree of stress of the subject, based on the static attribute information and the observation information.

### TECHNICAL FIELD

The present disclosure relates to a technical field of a stress estimation device, a stress estimation method, and a storage medium configured to perform processing related to estimation of a stress state.

### BACKGROUND

There is a device or a system for determining a stress state of a subject based on data measured from the subject. For example, Patent Literature 1 discloses a portable stress measuring device which determines a temporary stress degree of a subject on each day based on inspection data of the subject.

### CITATION LIST

### Patent Literature

Patent Literature 1: JP 2007-275287A

### SUMMARY

### Problem to be Solved

In the case of providing a service to estimate the degree of stress of a user and present the estimation result, high estimation accuracy is required to cause the user to continue the service. The “high estimation accuracy” herein includes at least one of outputting an estimation result having a high accuracy rate and outputting the estimation result on a scale of three or more. According to Patent Literature 1, the degree of stress is determined on a scale of two and therefore precise estimation of the stress state is not performed.

In view of the above-described issues, it is an object of the present disclosure to provide a stress estimation device, a stress estimation method, and a storage medium capable of accurately estimating a stress state.

### Means for Solving the Problem

In one aspect of the stress estimation device, there is provided a stress estimation device including:
a static attribute information acquisition means configured to acquire static attribute information regarding a static attribute of a subject;
an observation information acquisition means configured to acquire observation information that is information obtained by observing the subject; and
a stress estimation means configured to calculate a stress estimate value, which is an estimate value representing a degree of stress of the subject, based on the static attribute information and the observation information.

In one aspect of the stress estimation method, there is provided a stress estimation method executed by a computer, the control method including:
acquiring static attribute information regarding a static attribute of a subject;
acquiring observation information that is information obtained by observing the subject; and
calculating a stress estimate value, which is an estimate value representing a degree of stress of the subject, based on the static attribute information and the observation information.

In one aspect of the storage medium, there is provided a storage medium storing a program executed by a computer, the program causing the computer to:
acquire static attribute information regarding a static attribute of a subject;
acquire observation information that is information obtained by observing the subject; and
calculate a stress estimate value, which is an estimate value representing a degree of stress of the subject, based on the static attribute information and the observation information.

### Effect

An example advantage according to the present invention is to estimate a stress state of a subject with high accuracy.

### BRIEF DESCRIPTION OF THE DRAWINGS

FIG. 1 It illustrates a schematic configuration of a stress estimation system according to a first example embodiment.

FIG. 2 It illustrates an example of a hardware configuration of a stress estimation device common to each example embodiment.

FIG. 3 It is an example of a functional block diagram of the stress estimation device according to the first example embodiment.

FIG. 4 It is a diagram showing an outline of a stress estimation model.

FIG. 5A illustrates an example of a training data set for a stress estimation model.

FIG. 5B illustrates an example of input data to the stress estimation model.

FIG. 6 It is an example of a flowchart executed by the stress estimation device in the first example embodiment.

FIG. 7 It is a diagram showing an outline of processing in the stress estimation device according to a modification.

FIG. 8 It illustrates a schematic configuration of a stress estimation system according to a second example embodiment.

FIG. 9 It is a block diagram of a stress estimation device according to a third example embodiment.

FIG. 10 It illustrates an example of a flowchart executed by the stress estimation device according to the third example embodiment.

### EXAMPLE EMBODIMENTS

Hereinafter, example embodiments of a stress estimation device, a stress estimation method, and a storage medium will be described with reference to the drawings.

### First Example Embodiment

### (1) System Configuration

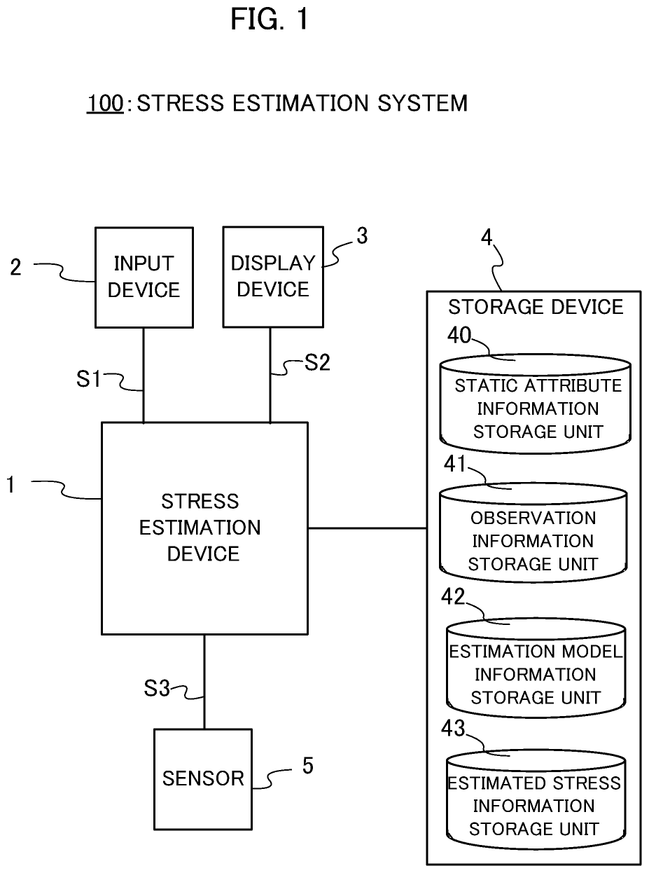

FIG. 1 shows a schematic configuration of a stress estimation system 100 according to a first example embodiment. The stress estimation system 100 estimates the stress of a subject and visualizes the estimation result. Examples of the “subject” herein include a sports player or an employee whose stress state is managed by the organization, and an individual user.

The stress estimation system 100 mainly includes a stress estimation device 1, an input device 2, a display device 3, a storage device 4, and a sensor 5.

The stress estimation device 1 performs data communication with the input device 2, the display device 3, and the sensor 5 through a communication network or through wireless or wired direct communication. The stress estimation device 1 estimates a stress state (specifically, a stress value representing a degree of stress) of the subject, based on the input signal “S1” supplied from the input device 2, the sensor signal “S3” supplied from the sensor 5, and information stored in the storage device 4. In addition, the stress estimation device 1 generates a display signal “S2” based on the estimation result of the stress state of the subject, and supplies the generated display signal S2 to the display device 3. The stress estimated by the stress estimation device 1 may be a short-term stress that is a stress in a relatively short term (within a range between a few minutes and a day), a chronic stress that is a stress in a long-term (chronic) on a weekly or monthly basis, or may be both of the short-term stress and the chronic stress.

The input device 2 is one or more interfaces configured to accept user input (manual input) of information regarding each subject. The user who performs input of information using the input device 2 may be the subject itself, or may be a person who manages or supervises the activity of the subject. The input device 2 may be a variety of user input interfaces such as, for example, a touch panel, a button, a keyboard, a mouse, and a voice input device. The input device 2 supplies the input signal S1 generated based on the input from the user to the stress estimation device 1. The display device 3 displays information based on the display signal S2 supplied from the stress estimation device 1. Examples of the display device 3 include a display and a projector.

The sensor 5 measures a biological signal or the like of the subject and supplies the measured biological signal or the like to the stress estimation device 1 as a sensor signal S3. In this instance, the sensor signal S3 may be any biological signal (including vital information) such as heartbeat, EEG, amount of perspiration, amount of hormonal secretion, cerebral blood flow, blood pressure, body temperature, electromyogram, electrocardiogram, respiration rate, pulse wave, acceleration. The sensor 5 may be a device that analyzes blood of the subject and outputs the analysis result as a sensor signal S3. The sensor 5 may be a wearable terminal worn by the subject, or may be a camera for photographing the subject or a microphone for generating a voice signal of the subject's utterance. The sensor 5 may be a terminal such as a personal computer and a smartphone operated by the subject. In this instance, the sensor 5 may supply information corresponding to the operation quantity of the personal computer or the smart phone to the stress estimation device 1 as the sensor signal S3.

The storage device 4 is a memory configured to store various information necessary for estimating the stress state. The storage device 4 may be an external storage device, such as a hard disk, connected to or embedded in the stress estimation device 1, or may be a storage medium, such as a flash memory. The storage device 4 may be a server device configured to perform data communication with the stress estimation device 1. Further, the storage device 4 may be configured by a plurality of devices.

The storage device 4 includes a static attribute information storage unit 40, an observation information storage unit 41, an estimation model information storage unit 42, and an estimation stress information storage unit 43.

The static attribute information storage unit 40 stores static attribute information, which is information indicating the static attribute of the subject (i.e., the information that hard to vary over time or that varies over time regularly). For example, the static attribute information is information regarding the gender, age, personality, or perception tendency of the subject, or a combination thereof. The static attribute information may be generated by the stress estimation device 1 and stored in the storage device 4, or may be generated in advance by a device other than the stress estimation device 1 and stored in the storage device 4. For example, the static attribute information is generated based on the answer result (i.e., subjective measurement result) of a questionnaire by the subject. For example, as a questionnaire to measure the personality of a subject, the Big Five personality test is known. The answer result of the questionnaire is an example of the subjective information by a subject. For example, the static attribute information of each subject is stored in the static attribute information storage unit 40 in association with the identification information (subject ID) of the each subject.

The observation information storage unit 41 stores observation information regarding the subject generated on the basis of the sensor signal S3 acquired by the stress estimation device 1 from the sensor 5. For example, the observation information stored in the observation information storage unit 41 is information in which the sensor signal S3 collected for each subject is associated with the identification information of the subject (subject ID) and the date and time information related to the generation or reception of the sensor signal S3. In this case, the observation information may include any information correlated with stress, examples of which is include: any biological signal of the subject (including vital information) such as heart rate, EEG, pulse wave, sweating amount, hormonal output, cerebral blood flow, blood pressure, body temperature, myoelectricity, respiration rate, and acceleration regarding the subject; image or voice data of the subject; and information regarding the operating status of the terminal used by the subject. The observation information may also include biological data (including sleep time) obtained by observing the subject during sleeping of the subject.

The estimation model information storage unit 42 stores information on the stress estimation model that is a model configured to calculate an estimate value of the stress of the subject. In this case, for example, the stress estimation model is a model trained to output a stress estimate value of a subject when feature values of static attribute information and feature values of observation information regarding the subject are inputted to the model. Here, the stress estimation model may be any machine learning model (including a statistical model) such as a neural network, a support vector machine, and the like. The estimation model information storage unit 42 stores parameter information necessary for configuring the stress estimation model. For example, when the stress estimation model is a model based on a neural network such as a convolutional neural network, the estimation model information storage unit 42 stores information regarding various parameters such as the layer structure, the neuron structure of each layer, the number of filters and the filter size in each layer, and the weight for each element of each filter.

The estimated stress information storage unit 43 stores the estimated stress information regarding the stress value (also referred to as “stress estimate value”) of the subject estimated by the stress estimation device 1 The estimated stress information is, for example, a database having records each of which associates the stress estimate value calculated by the stress estimation device 1 with the date and time information indicating the estimation date and time and the identification information (subject ID) of the subject. The above-described “estimation date and time” may be the generation date and time of the signal used for the estimation, or may be the date and time when the estimation was performed.

The configuration of the stress estimation system 100 shown in FIG. 1 is an example, and various changes may be made to the configuration. For example, the input device 2 and the display device 3 may be configured integrally. In this case, the input device 2 and the display device 3 may be configured as a tablet-type terminal that is incorporated into or separate from the stress estimation device 1. Further, the input device 2 and the sensor 5 may be configured integrally. Further, the stress estimation device 1 may be configured by a plurality of devices. In this case, the plurality of devices constituting the stress estimation device 1 performs transmission and reception of information necessary for executing the process allocated in advance among the plurality of devices. In this case, the stress estimation device 1 functions as an information processing system.

### (2) Hardware Configuration of Stress Estimation Device

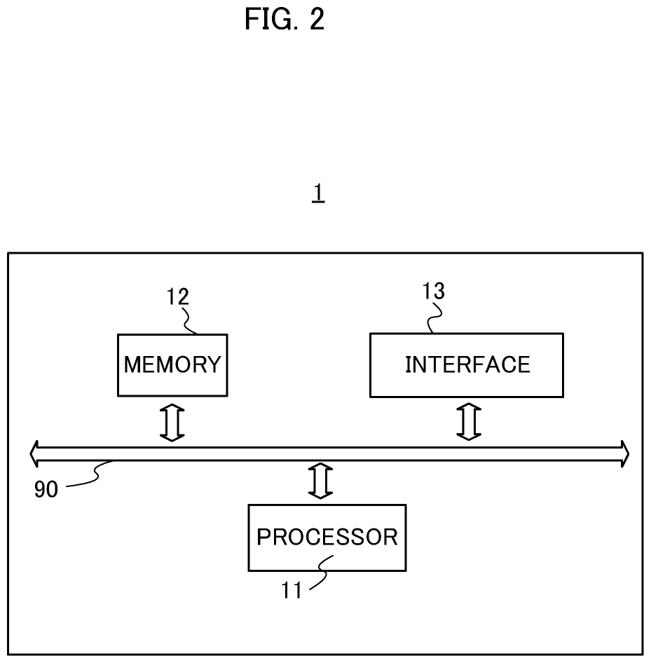

FIG. 2 shows a hardware configuration of the stress estimation device 1. The stress estimation device 1 includes a processor 11, a memory 12, and an interface 13 as hardware. The processor 11, memory 12 and interface 13 are connected to one another via a data bus 90.

The processor 11 functions as a controller (arithmetic unit) that performs overall control of the stress estimation device 1 by executing a program stored in the memory 12. Examples of the processor 11 include a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by a variety of volatile and non-volatile memories, such as a RAM (Random Access Memory), a ROM (Read Only Memory), and a flash memory. Further, a program for the stress estimation device 1 to execute a process is stored in the memory 12. A part of the information stored in the memory 12 may be stored in one or more external storage devices capable of communicating with the stress estimation device 1, or may be stored in a storage medium detachable from the stress estimation device 1.

The interface 13 is one or more interfaces for electrically connecting the stress estimation device 1 to other devices. Examples of these interfaces include a wireless interface, such as a network adapter, for transmitting and receiving data to and from other devices wirelessly, and a hardware interface, such as a cable, for connecting to other devices.

The hardware configuration of the stress estimation device 1 is not limited to the configuration shown in FIG. 2. For example, the stress estimation device 1 may include at least one of the input device 2 and the display device 3. In addition, the stress estimation device 1 may be connected to or incorporate an audio output device such as a speaker.

### (3) Stress Estimation Process

Next, the stress estimation process performed by the stress estimation device 1 will be described. Schematically, the stress estimation device 1 calculates the stress estimate value of the subject using both the static attribute information regarding the subject and the observation information regarding the subject. Thereby, the stress estimation device 1 estimates the stress of the subject with high accuracy and presents the estimation result.

(3-1) Functional Blocks

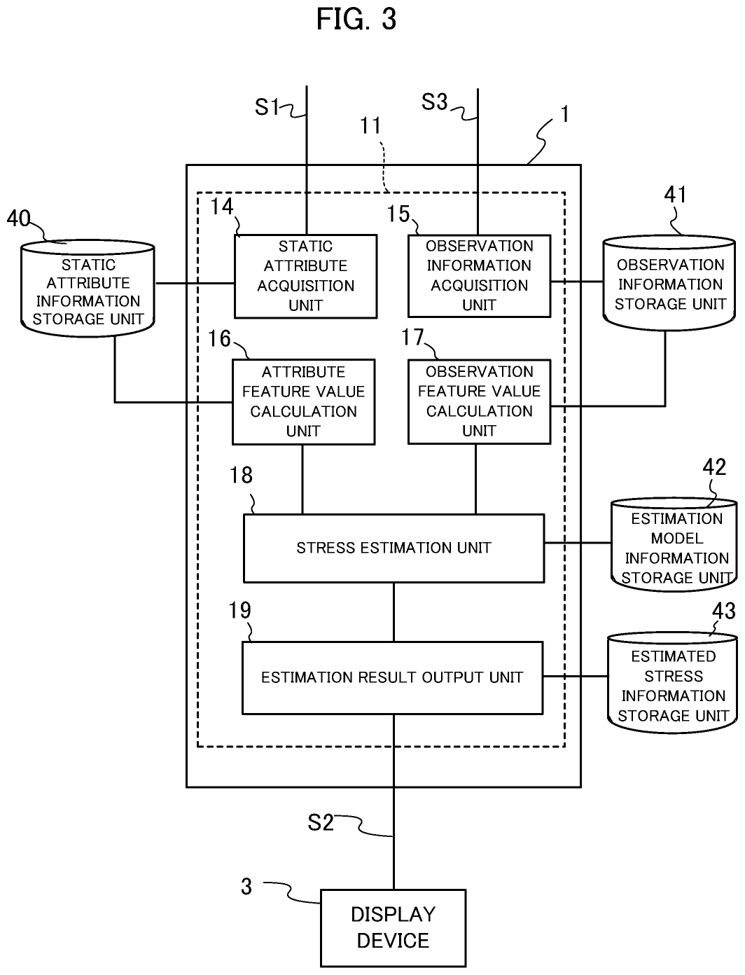

FIG. 3 is an example of a functional block diagram of the stress estimation device 1. The processor 11 of the stress estimation device 1 functionally includes a static attribute acquisition unit 14, an observation information acquisition unit 15, an attribute feature value calculation unit 16, an observation feature value calculation unit 17, a stress estimation unit 18, and an estimation result output unit 19. In FIG. 3, any blocks to exchange data with each other are connected by a solid line, but the combination of the blocks to exchange data with each other is not limited to the combination shown in FIG. 3. The same applies to the drawings of other functional block diagrams described below.

The static attribute acquisition unit 14 acquires the static attribute information regarding the subject based on the input signal S1. In this instance, for example, the static attribute acquisition unit 14 displays an input screen image regarding a questionnaire on the display device 3 and acquires the input signal S1 indicating the answer of the questionnaire inputted on the input screen image as the static attribute information. The questionnaire adopted in this case is a questionnaire for measuring a static attribute such as gender, age, personality, and tendency of cognition. The static attribute acquisition unit 14 stores the acquired static attribute information in the static attribute information storage unit 40 in association with the identification information of the subject and the information indicating the measurement date and time. The static attribute acquisition unit 14 may acquire the static attribute information at least once for each subject, and need not acquire each time the stress is estimated.

The observation information acquisition unit 15 generates observation information regarding the subject based on the sensor signal S3, and stores the observation information in the observation information storage unit 41. In this instance, the observation information acquisition unit 15 stores observation information in which the sensor signal S3 is associated with the identification information regarding the subject and information indicating the observation date and time in the observation information storage unit 41.

The attribute feature value calculation unit 16 acquires the static attribute information regarding the subject from the static attribute information storage unit 40 at the timing of stress estimation regarding the subject and extracts feature values (also referred to as “attribute feature values”) of the acquired static attribute information. In this case, the attribute feature value calculation unit 16 extracts the feature values related to the stress (i.e., correlates with the stress value) from the static attribute information as the attribute feature values.

For example, when the static attribute information indicates the answer result of a questionnaire, the attribute feature value calculation unit 16 extracts the scores of items related to the stress estimation as the attribute feature values. For example, when the static attribute information indicates the answer result of the Big Five personality test, the highest scores (e.g., top one or two scores) in order of correlation with stress among various scores calculated as a result of the Big Five personality test are used as the attribute feature values. In another example, when the static attribute information indicates one or more attributes such as the age or gender of the subject, the attribute feature value calculation unit 16 extracts numerical values representing the classes (categories) of the attributes of the subject as the attribute feature values. The attribute feature values extracted by the attribute feature value calculation unit 16 are expressed as a feature vector with a predetermined number of dimensions.

The timing of stress estimation of the subject may be a timing requested by the user based on the input signal S1, or may be a predetermined timing.

At the timing of the stress estimation of the subject, the observation feature value calculation unit 17 acquires, from the observation information storage unit 41, the observation information regarding the subject observed during a target period of the stress estimation, and extracts the feature values (also referred to as “observation feature values”) of the acquired observation information. For example, when the observation information is the perspiration data of the subject, the observation feature value calculation unit 17 extracts the statistical values such as the average value, the maximum value, and the like of the perspiration amount (which may be normalized for each user) in the target period of the stress estimation as the observation feature values. The target period of the stress estimation is, for example, a period of time corresponding to a predetermined number of days just before the timing of the stress estimation, and the predetermined number of days described above is determined according to the type (e.g., chronic stress, short-term stress) of stress to be estimated. The observation feature values extracted by the observation feature value calculation unit 17 are expressed as a feature vector with a predetermined number of dimensions.

The stress estimation unit 18 calculates the stress estimate value of the subject based on the attribute feature values supplied from the attribute feature value calculation unit 16 and the observation feature values supplied from the observation feature value calculation unit 17. In this case, the stress estimation unit 18 builds (configures) the trained stress estimation model by referring to the estimation model information storage unit 42, and acquires the stress estimate value by inputting the attribute feature values and the observation feature values into the stress estimation model. The stress estimation unit 18 supplies the calculated stress estimate value to the estimation result output unit 19.

The estimation result output unit 19 outputs information based on the stress estimate value supplied from the stress estimation unit 18. For example, the estimation result output unit 19 stores, in the estimated stress information storage unit 43, the stress estimate value supplied from the stress estimation unit 18 in association with the identification information regarding the subject or the like. Further, the estimation result output unit 19 generates a display signal S2 for displaying information regarding the stress estimate value, and then supplies the display device 3 with the display signal S2, thereby to display the information regarding the stress estimate value on the display device 3. The information regarding the stress estimate value may be the stress estimate value itself, or may be information regarding the level of the stress determined based on the comparison between the stress estimate value and a predetermined threshold, or may be information regarding the advice according to the level. The viewer of the display device 3 in this case, for example, may be the subject, or may be a person who manages or supervises the subject. The estimation result output unit 19 may also perform audio output of information regarding the stress estimate value by an audio output device (not shown).

Each component of the static attribute acquisition unit 14, the observation information acquisition unit 15, the attribute feature value calculation unit 16, the observation feature value calculation unit 17, the stress estimation unit 18, and the estimation result output unit 19 described in FIG. 3 can be realized by the processor 11 executing a program, for example. Additionally, the necessary programs may be recorded on any non-volatile storage medium and installed as necessary to realize each component. It should be noted that at least a portion of each of these components may be implemented by any combination of hardware, firmware, and software, without being limited to being implemented by software based on a program. At least some of these components may also be implemented using user programmable integrated circuit such as, for example, a FPGA (Field-Programmable Gate Array) and a microcontroller. In this case, the integrated circuit may be used to realize a program functioning as each of the above components. Further, at least a part of the components may be configured by ASSP (Application Specific Standard Produce), ASIC (Application Specific Integrated Circuit) or a quantum processor (quantum computer control chip). Thus, each of the above-described components may be realized by various hardware. Furthermore, each of these components may be implemented by cooperation of a plurality of computers, for example, using cloud computing technology. The above is true for other example embodiments described later.

(3-2) Stress Estimation Model

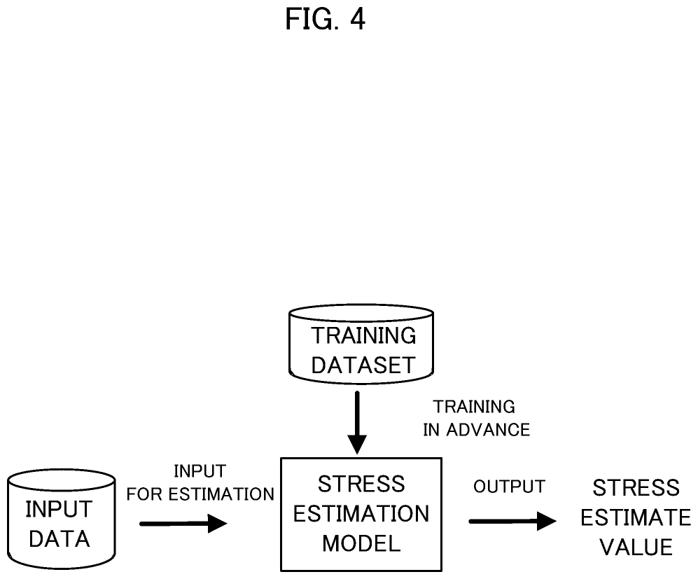

FIG. 4 is a diagram illustrating an outline of a stress estimation model to be used by the stress estimation unit 18. The stress estimation model is trained in advance based on the training data set prepared in advance, and the learned parameters and the like are stored in advance in the estimation model information storage unit 42. In this case, the training data set includes a plurality of sets of input data that are attribute feature values and observation feature values to be inputted to the stress estimation model and the corresponding correct answer data (the stress estimate value to be used as the correct answer) that the stress estimation model should output when the corresponding input data is inputted thereto.

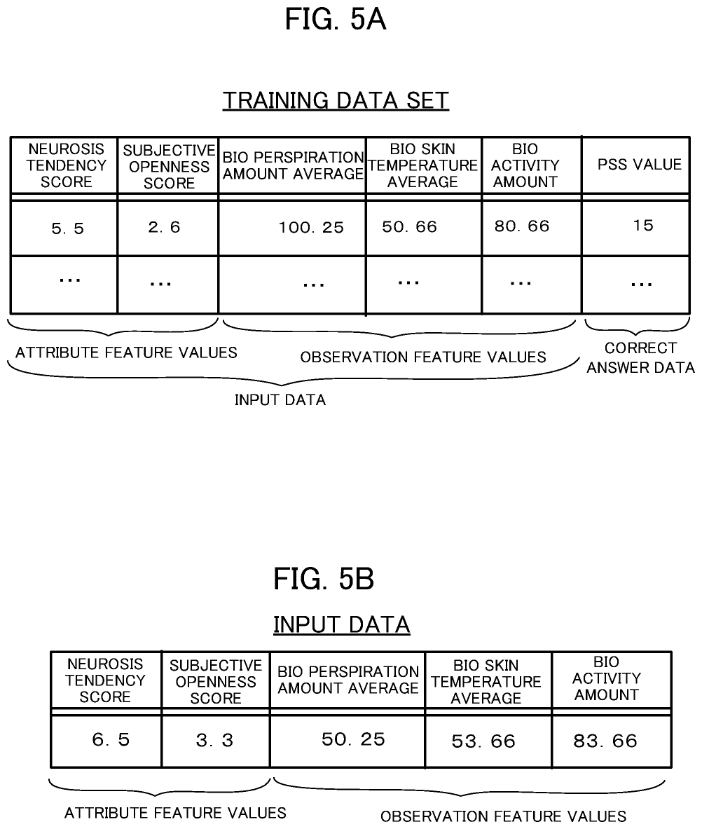

FIG. 5A shows an example of a training data set. As shown in FIG. 5A, the training data set includes a plurality of sets of two types of attribute feature values, three types of observation feature values, and the PSS (Perceived Stress Scale) which is used as the correct answer data.

In FIG. 5A, the “subjective neurosis tendency score” and the “subjective openness score” are included as attribute feature values. Here, the “subjective neurosis tendency score” and the “subjective openness score” are scores (indices) which are calculated based on the questionnaire results in the Big Five personality test, and they are examples of scores (indices) deeply related to stress among the scores calculated based on the questionnaire result in the Big Five personality test. It is noted that “extroversion score” may be used in place of the “subjective openness score” or only the “subjective neurosis tendency score” may be used depending on the type of the population.

As another example of the attribute feature values which are deeply related to stress, there is known an index on resilience. There are various definitions of resilience, and the score (indices) of resilience according to each definition can be measured based on the answer result of questionnaire for each definition. For example, resilience is defined as a process, ability, or outcome to adapt a difficult or threatening situation. The resilience may also be classified into a primary resilience (the ability to maintain psychological health even when exposed to stressors) and a secondary resilience (the ability to overcome and restore to a healthy state even after being temporarily in an inappropriate state).

As yet other examples of the attribute feature value that is deeply related to stress, various indices relating to the personality may be used, such as the MPI (Maudsley Personality Inventory), the EPP (Eysenck Personality Profiler), the YG personality test. In addition, examples of the personality test include Egograms, EQ test, SPI, and new edition TEG 3, and any stress-related results selected from the above-mentioned personality test results may be used as the attribute feature values.

As the observation feature values, there are included “bio perspiration amount average”, “bio skin temperature average” and “bio activity amount”. The “bio perspiration amount average” is an average value of the amount of perspiration measured during a predetermined period by a wearable terminal or the like for measuring the amount of perspiration of a subject. Further, the “bio skin temperature average” is an average value of the skin temperature measured during a predetermined period by a wearable terminal or the like for measuring the skin temperature of a subject. The “bio activity amount” is the average value or total value of the amount of activity of a subject specified from the acceleration or the like measured by a wearable terminal or the like for measuring the subject's acceleration. It is noted that the “bio perspiration amount average”, “bio skin temperature average” and “bio activity amount” are just a part of examples of the observation feature values and any feature values calculated from biological signals such as perspiration data, skin temperature data, and acceleration data may be adopted as the observation feature values.

The index to be used as the attribute feature value and the observation feature value may be selected such that the absolute value of the correlation between the index and the PSS value that is used as the correct answer data of the stress is approximately 0.3 (e.g., 0.1 to 0.3).

The training data set shown in FIG. 5A includes the “PSS value” as correct answer data of the stress value to be estimated. Here, the PSS value is calculated from the answer to the PSS questionnaire for measuring the dynamical stress which varies over time.

Then, in training the stress estimation model, each record of the training data set is extracted in order, and the parameters of the stress estimation model are updated by use of the each record. In this case, the parameters of the stress estimation model are determined so that the error (loss) between the estimation result outputted by the stress estimation model to which the attribute feature values and the observation feature values are inputted and the PSS value which is the correct answer data is minimized. The algorithm for determining parameters to minimize the loss may be any learning algorithm used in machine learning, such as the gradient descent method and the error back propagation method.

FIG. 5B shows an example of input data that is inputted at the time of stress estimation to the stress estimation model trained by the training data set shown in FIG. 5A. As shown in FIG. 5A and FIG. 5B, the input data to be inputted to the stress estimation model includes the attribute features and the observed features “subjective neurosis score”, “subjective openness score”, “bio perspiration amount average”, “bio skin temperature average” and “bio activity amount”. Then, the stress estimation unit 18 inputs the input data to the stress estimation model configured by referring to the estimation model information storage unit 42, and acquires the stress estimate value outputted from the stress estimation model in that case. Here, the stress estimate value outputted from the stress estimation model have the same value range as the PSS value. Thus, the stress estimation unit 18 can calculate the estimated stress value from the attribute feature values and the observation feature values with high accuracy.

The stress estimation model need only be configured to output a value on a scale of two or more as the stress estimate value, and need not be configured to output a continuous value as the stress estimate value. For example, if the stress estimation model is configured to output a stress estimate value on a scale of 1 to V (V is an integer of 3 or more), the PSS value is converted into a value on the scale of 1 to V in the learning stage, and the stress estimation model is trained by using the value on the scale of 1 to V as the correct answer data. By using such a stress estimation model, the stress estimation unit 18 acquires a stress estimate value on the scale of 1 to V. In another example, the stress estimation unit 18 may use a stress estimation model configured to output a continuous value. After acquiring the continuous value outputted by the stress estimation model, the stress estimation unit 18 converts the continuous value to the value on the scale of 1 to V, and supply the value on the scale of 1 to V to the estimation result output unit 19.

(3-3) Processing Flow

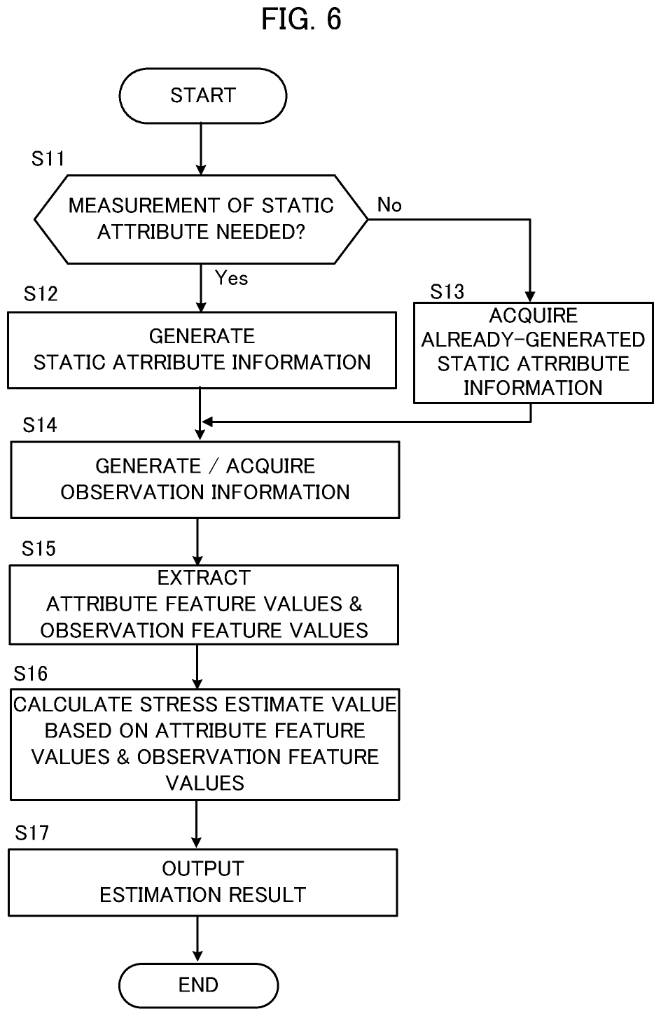

FIG. 6 is an example of a flowchart that is executed by the stress estimation device 1 in the first example embodiment. For example, the stress estimation device 1 executes a process of the flowchart shown in FIG. 6, when it is determined that it is a predetermined timing of stress estimation.

First, the stress estimation device 1 determines whether or not to measure a static attribute of the subject (step S11). In this case, the stress estimation device 1 acquires the identification information regarding the subject by any authentication process, for example, and determines whether or not the static attribute information associated with the identification information regarding the subject is included in the static attribute information storage unit 40. The stress estimation device 1 determines that it is not necessary to acquire the static attribute of the subject if the corresponding static attribute information is included in the static attribute information storage unit 40. In contrast, stress estimation device 1 determines that it is necessary to acquire the static attribute of the subject if the corresponding static attribute information is not included in the static attribute information storage unit 40. The stress estimation device 1 may determine that the static attribute of the subject needs to be acquired again if the corresponding static attribute information is generated a predetermined time (for example, several years) or more before.

Then, when it is determined that it is necessary to measure the static attribute (step S11; Yes), the stress estimation device 1 performs a process of generating the static attribute information (step S12). In this case, for example, the stress estimation device 1 receives the answer of a questionnaire by displaying the input screen image of the questionnaire or the like, and generates the static attribute information based on the received answer of the questionnaire. On the other hand, when it is determined that it is necessary to measure the static attribute (step S11; No), the stress estimation device 1 acquires already-generated static attribute information regarding the subject from the static attribute information storage unit 40 (step S13).

Next, the stress estimation device 1 generates or acquires the observation information such as the biological data of the subject (step S14). In this instance, the stress estimation device 1 generates the most recent observation information regarding the subject based on the sensor signal S3 outputted by the sensor 5. In addition, when using the past observation information generated during the latest period with a predetermined time length, the stress estimation device 1 acquires the corresponding observation information from the observation information storage unit 41.

Next, the stress estimation device 1 extracts the attribute feature values and the observation feature values (step S15). In this instance, the stress estimation device 1 extracts the attribute feature values from the static attribute information generated or acquired at step S12 or step S13, and extracts the observation feature values from the observation information generated or acquired at step S14.

Then, the stress estimation device 1 calculates the stress estimate value of the subject based on the attribute feature values and the observation feature values (step S16). In this instance, the stress estimation device 1 builds the stress estimation model by referring to the estimation model information storage unit 42, and acquires the stress estimate value from the stress estimation model by inputting the attribute feature values and the observation feature values extracted at step S15 into the stress estimation model. Then, the stress estimation device 1 outputs the estimation result of the stress state (step S17).

### (4) Modification

The stress estimation device 1 may selectively use a plurality of stress estimation models based on the attribute feature values.

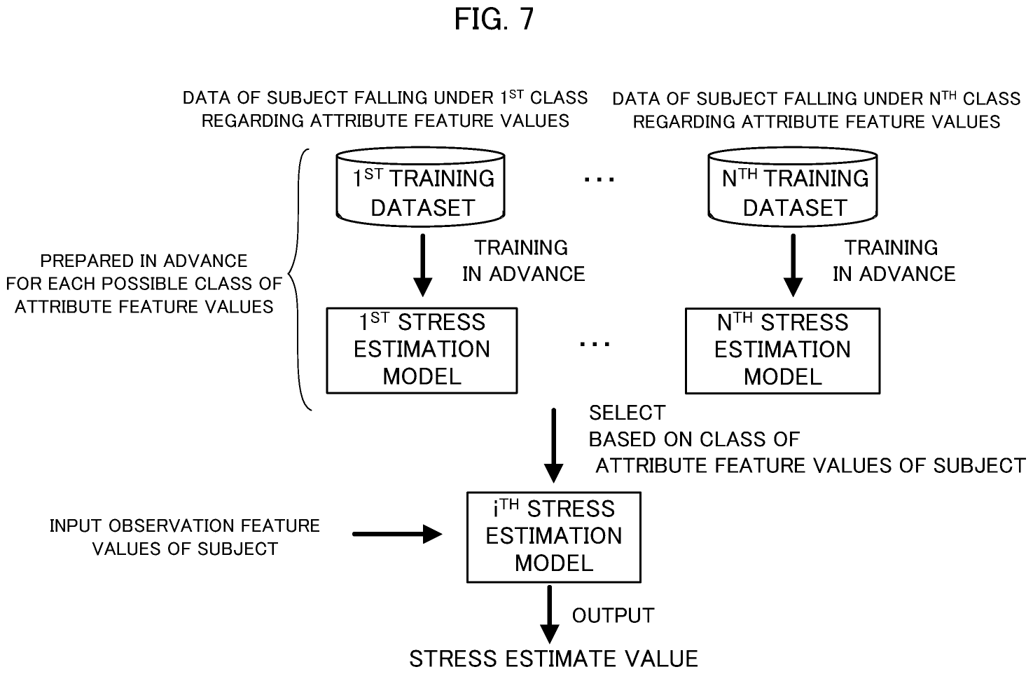

FIG. 7 is a diagram showing an outline of the process executed by the stress estimation device 1 according to the modification. In the example shown in FIG. 7, N (N is an integer of 2 or more) stress estimation models (first stress estimation model to N-th stress estimation model) are prepared in advance for possible classes of the attribute feature values, respectively, and the stress estimation device 1 selects a stress estimation model (i-th stress estimation model in this case) to be used based on the class of the attribute feature values of the subject.

Here, the first stress estimation model to the N-th stress estimation model are trained using the first training data set to the N-th training data set prepared for possible classes of the attribute feature values, respectively. For example, the first stress estimation model is preliminarily trained by use of a first training data set having a plurality of records each including observation data of subject(s) whose attribute feature values fall under a first class and the corresponding correct data of the stress (e.g., the PSS value based on a questionnaire). Each of the other stress estimation models is also trained in advance by use of a training data set having a plurality of records each including observation information regarding subject(s) whose attribute feature values fall under each corresponding class and the correct answer data of stress. Then, the learned parameters of the first stress estimation model to the N-th stress estimation model are stored in advance in the estimation model information storage unit 42.

Then, in estimating the stress of a subject, the stress estimation device 1 determines whether or not the class of the attribute feature values of the subject corresponds to any of the first class to the N-th class, and selects the i-th stress estimation model corresponding to the determined class (here, the i-th class). Then, the stress estimation device 1 builds the i-th stress estimation model with reference to the estimation model information storage unit 42 and inputs the observation feature values and the attribute feature values (or only the observation feature values) regarding the subject to the i-th stress estimation model. When the number of elements of the attribute does not match the number N, like the classification of male and female, the i-th stress estimation model may include multiple attributes and the attribute feature values are also used after classification in this case. Thus, the stress estimation device 1 acquires the stress estimate value outputted by the i-th stress estimation model.

Thus, the stress estimation device 1 according to this modification can accurately estimate the stress of the subject even when a plurality of stress estimation models is selectively used based on the attribute feature values.

### Second Example Embodiment

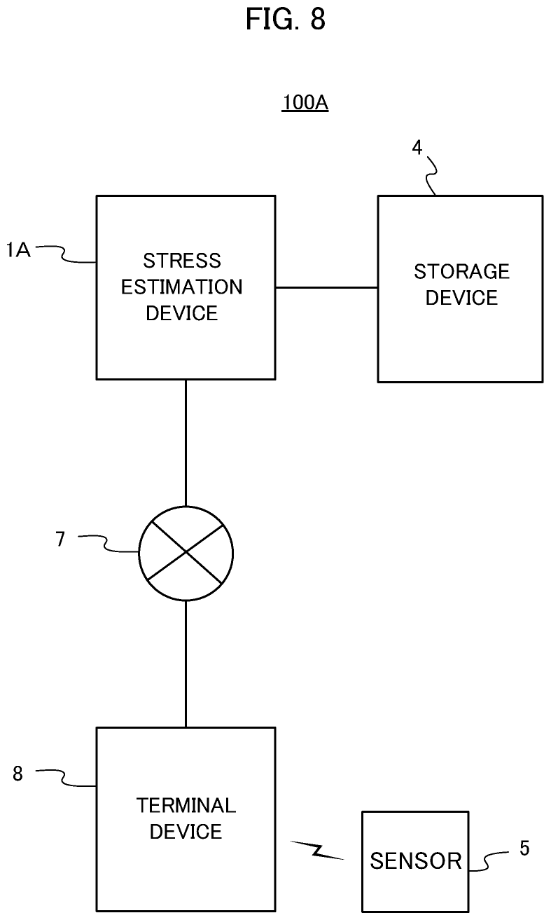

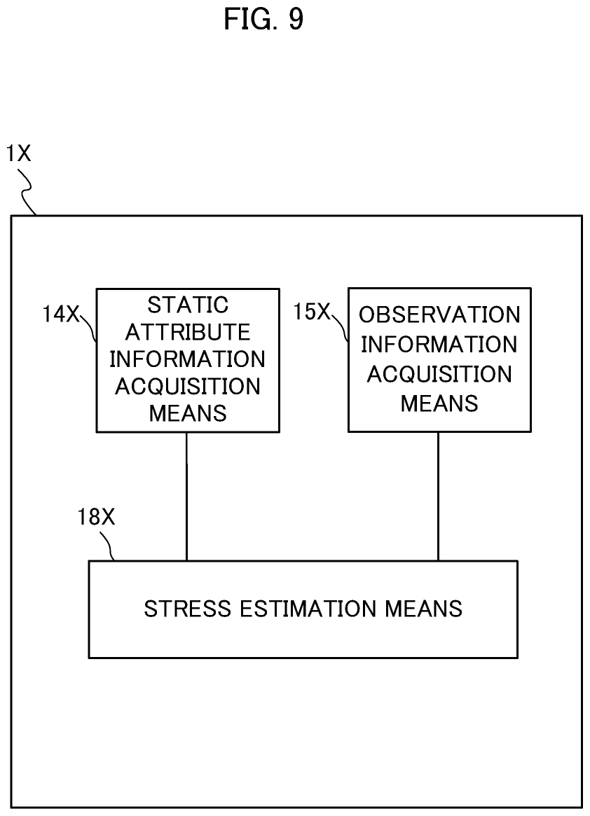

FIG. 8 shows a schematic configuration of a stress estimation system 100A according to a second example embodiment. The stress estimation system 100A according to the second example embodiment is a system according to a server client model, and a stress estimation device 1A functioning as a server device performs the same process as the stress estimation device 1 according to the first example embodiment. Hereinafter, the same components as those in the first example embodiment are denoted by the same reference numerals, and a description thereof will be omitted accordingly.

As shown in FIG. 8, the stress estimation system 100A mainly includes a stress estimation device 1A that functions as a server, a storage device 4, and a terminal device 8 that functions as a client. The stress estimation device 1A and the terminal device 8 perform data communication with each other via the network 7.

The terminal device 8 is a terminal used by a subject as a user, and is equipped with an input function, a display function, and a communication function, and functions as an input device 2 and the display device 3 shown in FIG. 1. Examples of the terminal device 8 include a personal computer, a tablet-type terminal such as a smartphone, and a PDA (Personal Digital Assistant). The terminal device 8 is electrically connected to the sensor 5 such as a wearable sensor worn by a user, and transmits a biological signal or the like (i.e., information corresponding to the sensor signal S3 in FIG. 1) of a subject outputted by the sensor 5 to the stress estimation device 1A. Further, the terminal device 8 receives the user input or the like relating to the answer of a questionnaire, and transmits the information (information corresponding to the input signal S1 in FIG. 1) generated by the user input to the stress estimation device 1A.

The stress estimation device 1A has the same hardware configuration as the hardware configuration of the stress estimation device 1 shown in FIG. 2, and the processor 11 of the stress estimation device 1A has the functional blocks shown in FIG. 3. Then, the stress estimation device 1A receives the information corresponding to the input signal S1 and the sensor signal S3 in FIG. 1 from the terminal device 8 via the network 7, and executes the stress estimation process. The stress estimation device 1A transmits an output signal for outputting the stress estimation result to the terminal device 8 through the network 7 based on the display request from the terminal device 8.

According to the second example embodiment, it is possible to estimate the stress state of the subject based on the biological signal of the subject or the questionnaire result received from the terminal used by the subject, and suitably present the estimation result to the subject on the terminal.

### Third Example Embodiment

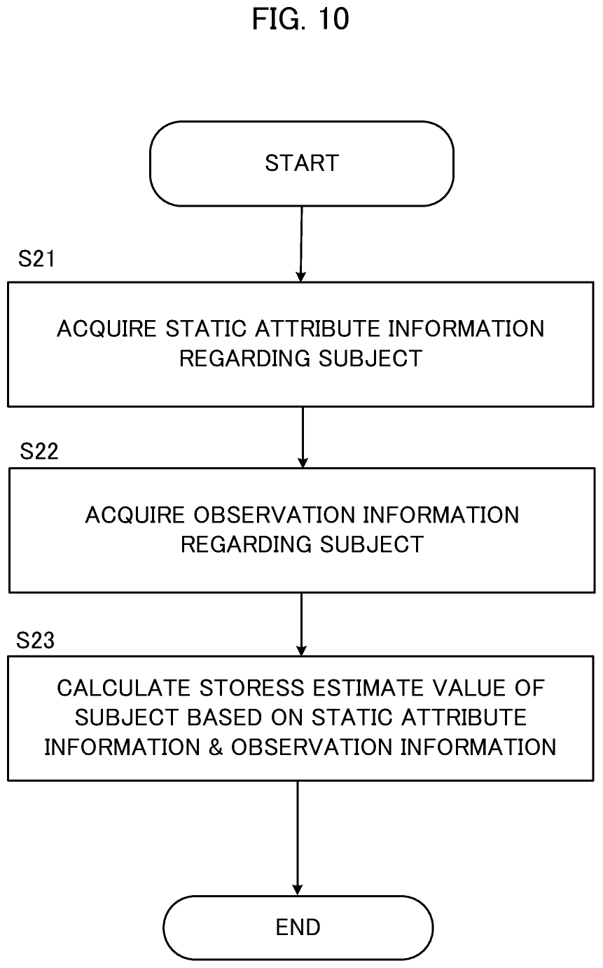

FIG. 10 is a block diagram of a stress estimation device 1X according to the third example embodiment. The stress estimation device 1X mainly includes a static attribute information acquisition means 14X, an observation information acquisition means 15X, and a stress estimation means 18X. The stress estimation device 1X may be configured by a plurality of devices.

The static attribute information acquisition means 14X is configured to acquire static attribute information regarding a static attribute of a subject. In this case, the static attribute information acquisition means 14X may generate the static attribute information by receiving the answer of a questionnaire from the subject, or may acquire the static attribute information regarding the subject stored in the storage device or the like in advance. Examples of the static attribute information acquisition means 14X in the former case include the static attribute acquisition unit 14 in the first example embodiment or the second example embodiment.

The observation information acquisition means 15X is configured to acquire observation information that is information (in other words, objectively-measured information) obtained by observing the subject. In this instance, the observation information acquisition means 15X may generate observation information by receiving a signal from a sensor that senses the subject, or may acquire observation information of the subject stored in advance in a storage device or the like. Examples of the observation information acquisition means 15X in the former case include the observation information acquisition unit 15 in the first example embodiment or the second example embodiment.

The stress estimation means 18X is configured to calculate a stress estimate value, which is an estimate value representing a degree of stress of the subject, based on the static attribute information and the observation information. Examples of the stress estimation means 18X include the stress estimation unit 18 (or a combination of the attribute feature value calculation unit 16, the observation feature value calculation unit 17, and the stress estimation unit 18) according to the first example embodiment or the second example embodiment.

FIG. 10 is an exemplary flowchart that is executed by the stress estimation device 1X in the third example embodiment. First, the static attribute information acquisition means 14X acquires static attribute information regarding a static attribute of a subject (step S21). Then, the observation information acquisition means 15X acquires observation information that is information obtained by observing the subject (step S22). The stress estimation means 18X calculates a stress estimate value, which is an estimate value representing a degree of stress of the subject, based on the static attribute information and the observation information (step S23).

According to the third example embodiment, the stress estimation device 1X can accurately estimate the stress state of the subject by considering both the static attribute information and the observation information regarding the subject.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### Supplementary Note 1

A stress estimation device comprising:
a static attribute information acquisition means configured to acquire static attribute information regarding a static attribute of a subject;
an observation information acquisition means configured to acquire observation information that is information obtained by observing the subject; and
a stress estimation means configured to calculate a stress estimate value, which is an estimate value representing a degree of stress of the subject, based on the static attribute information and the observation information.

### Supplementary Note 2

The stress estimation device according to Supplementary Note 1, further comprising:
an attribute feature value calculation means configured to calculate an attribute feature value that is a feature value of the static attribute information; and
an observation feature value calculation means configured to calculate an observation feature value that is a feature value of the observation information, wherein the stress estimation means is configured to calculate the stress estimate value based on the attribute feature value and the observation feature value.

### Supplementary Note 3

The stress estimation device according to Supplementary Note 2,
wherein the attribute feature value calculation means is configured to calculate an index relating to personality as the attribute feature value.

### Supplementary Note 4

The stress estimation device according to Supplementary Note 3,
wherein the attribute feature value calculation means is configured to calculate, as the index relating to personality, at least one of
   an index relating to a neurosis tendency of the subject,
   an index relating to extroversion of the subject,
   an index relating to openness of the subject, and
   an index relating to resilience of the subject.

### Supplementary Note 5

The stress estimation device according to any one of Supplementary Notes 1 to 4,
wherein the stress estimation means is configured to input data based on the static attribute information and the observation information regarding the subject to a stress estimation model,
the stress estimation model being trained to output, when input data based on static attribute information and observation information regarding the subject is inputted thereto, a stress estimate value of the subject.

### Supplementary Note 6

The stress estimation device according to any one of Supplementary Notes 1 to 4,
wherein the stress estimation means is configured to
   select, based on the static attribute information regarding the subject, a stress estimation model from stress estimation models trained for respective classes regarding the static attribute and
   input, to the selected stress estimation model, data based on the observation information regarding the subject, or, data based on the observation information and static attribute information regarding the subject,
the stress estimation models being trained to output, when input data based on observation information regarding the subject is inputted thereto, a stress estimate value of the subject.

### Supplementary Note 7

The stress estimation device according to any one of Supplementary Notes 1 to 6,
wherein the static attribute information includes subjective information generated by the subject.

### Supplementary Note 8

The stress estimation device according to any one of Supplementary Notes 1 to 7,
wherein the observation information includes biological data regarding the subject.

### Supplementary Note 9

The stress estimation device according to any one of Supplementary Notes 1 to 8, further comprising
an estimation result output means configured to display, or output by audio, information regarding an estimation result of the stress.

### Supplementary Note 10

A stress estimation method executed by a computer, the control method comprising:
acquiring static attribute information regarding a static attribute of a subject;
acquiring observation information that is information obtained by observing the subject; and
calculating a stress estimate value, which is an estimate value representing a degree of stress of the subject, based on the static attribute information and the observation information.

### Supplementary Note 11

A storage medium storing a program executed by a computer, the program causing the computer to:
acquire static attribute information regarding a static attribute of a subject;
acquire observation information that is information obtained by observing the subject; and
calculate a stress estimate value, which is an estimate value representing a degree of stress of the subject, based on the static attribute information and the observation information.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1. 1A, 1X Stress estimation device
2 Input device
3 Display device
4 Storage device
Sensor
8 Terminal device
100, 100A Stress estimation system

What is claimed is:

1. A stress estimation device comprising:
at least one memory configured to store instructions; and
at least one processor configured to execute the instructions to:
acquire static attribute information regarding a static attribute of a subject;
acquire observation information that is information obtained by observing the subject; and
calculate a stress estimate value, which is an estimate value representing a degree of stress of the subject, based on the static attribute information and the observation information.

2. The stress estimation device according to claim 1,
wherein the at least one processor is configured to further execute the instructions to:
calculate an attribute feature value that is a feature value of the static attribute information;
calculate an observation feature value that is a feature value of the observation information; and
calculate the stress estimate value based on the attribute feature value and the observation feature value.

3. The stress estimation device according to claim 2,
wherein the at least one processor is configured to execute the instructions to calculate an index relating to personality as the attribute feature value.

4. The stress estimation device according to claim 3,
wherein the at least one processor is configured to execute the instructions to calculate, as the index relating to personality, at least one of
an index relating to a neurosis tendency of the subject,
an index relating to extroversion of the subject,
an index relating to openness of the subject, and
an index relating to resilience of the subject.

5. The stress estimation device according to claim 1,
wherein the at least one processor is configured to execute the instructions input data based on the static attribute information and the observation information regarding the subject to a stress estimation model and obtain the stress estimate value from the stress estimation model,
the stress estimation model being trained to output, when input data based on static attribute information and observation information regarding the subject is inputted thereto, a stress estimate value of the subject.

6. The stress estimation device according to claim 1,
Wherein the at least one processor is configured to execute the instructions
select, based on the static attribute information regarding the subject, a stress estimation model from stress estimation models trained for respective classes regarding the static attribute and
input, to the selected stress estimation model, data based on the observation information regarding the subject, or, data based on the observation information and static attribute information regarding the subject and obtain the stress estimate value from the selected stress estimation model,
the stress estimation models being trained to output, when input data based on observation information regarding the subject is inputted thereto, a stress estimate value of the subject.

7. The stress estimation device according to claim 1,
wherein the static attribute information includes subjective information generated by the subject.

8. The stress estimation device according to claim 1,
wherein the observation information includes biological data regarding the subject.

9. The stress estimation device according to claim 1,
wherein the at least one processor is configured to further execute the instructions to display, or output by audio, information regarding an estimation result of the stress.

10. A stress estimation method executed by a computer, the control method comprising:
acquiring static attribute information regarding a static attribute of a subject;
acquiring observation information that is information obtained by observing the subject; and
calculating a stress estimate value, which is an estimate value representing a degree of stress of the subject, based on the static attribute information and the observation information.

11. A non-transitory computer readable storage medium storing a program executed by a computer, the program causing the computer to:
acquire static attribute information regarding a static attribute of a subject;
acquire observation information that is information obtained by observing the subject; and
calculate a stress estimate value, which is an estimate value representing a degree of stress of the subject, based on the static attribute information and the observation information.
